(19) United States
(12) Patent Application Publication
(10) Pub. No.: US 2004/0143227 A1
(43) Pub. Date: Jul. 22, 2004

(54) Thorax drainage system

(76) Inventors: Richard Rollin, Menzingen (CH); Beat Moser, Hunenberg (CH); Urs Koch, Greppen (CH)

Correspondence Address: MICHAEL H. BANIAK, BANIAK PINE & GANNON, Suite 1200, 150N. Wacker Drive, Chicago, IL, 60606, US

(21) Appl. No.: 10/735,084

(22) Filed: Dec. 12, 2003

(30) Foreign Application Priority Data
Jun. 5, 2002 (CH) 2002 0949/02

(51) Int. Cl. A61M 1/00

(52) U.S. Cl. 604/319

(57) ABSTRACT

The thorax drainage system consists of reusable containers, drainage line and connecting lines, and a vacuum pump with a battery for operating it, in which system at least the drainage line is a disposable line and all the components can be arranged on a mobile trolley.

### RELATED APPLICATIONS

This application is a continuation-in-part of U.S. Ser. No. ______, filed Dec. 12, 2003.

### FIELD OF THE INVENTION

The present invention related to a thorax drainage system.

### BACKGROUND OF THE INVENTION

The systems known today, which all operate with vacuum aspiration, are so constructed that most if not all of the system components (except the vacuum pump) are disposed of after being used once. A further disadvantage is that the drainage system is mains-operated, so that the patient is in practice confined to bed while the system is operating.

The thorax drainage system is of vital importance for the patient because, following surgical interventions in the thorax region, a vacuum has to be established and maintained in the thorax to ensure that the lungs can expand to the normal “operating size”.

### SUMMARY OF THE INVENTION

The object of the present invention is to make available a thorax drainage system which on the one hand satisfies the medical requirements and on the other hand offers the patient improved mobility (in order, among other things, to reduce the period spent confined to bed). At the same time, the system must be able to be used several times on the same patient (cost savings through reuse, also of the secretion-collecting container).

According to the invention, this object is achieved, in the present thorax drainage system, by the features set out in the appended claims.

An illustrative embodiment of the subject of the invention is explained in rather more detail below with reference to the drawing.

### BRIEF DESCRIPTION OF THE DRAWINGS

FIG. 1 shows a purely diagrammatic representation of the construction of the system according to the invention.

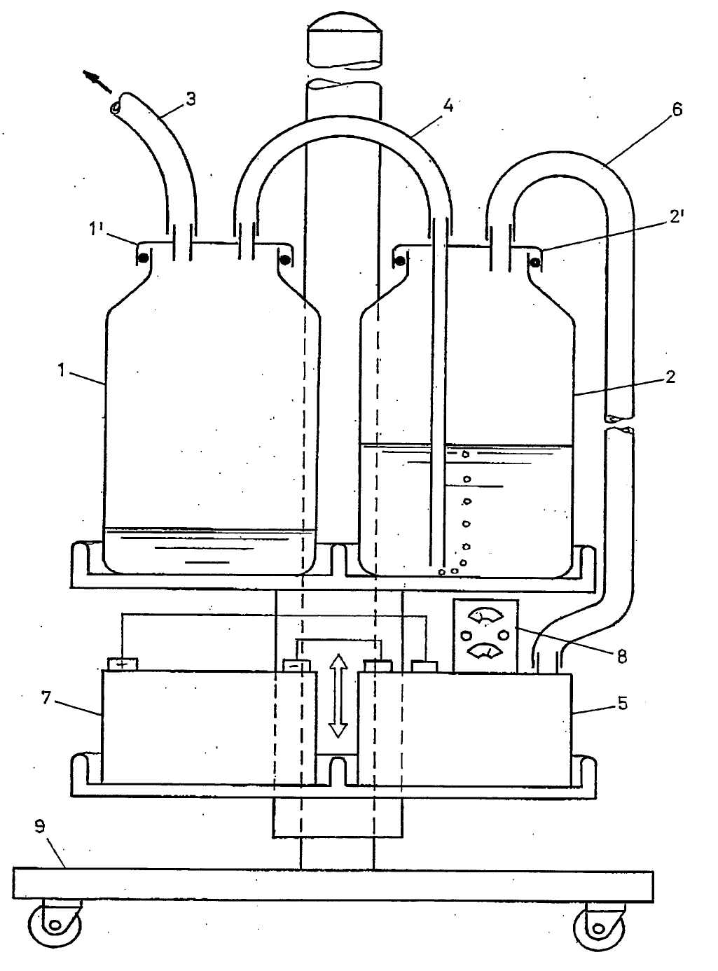

### DETAILED DESCRIPTION OF EMBODIMENTS OF THE INVENTION

The container forming the secretion-collecting chamber 1 is closed tight with a cover 1′. The drainage line 3 leading to the patient, and designed as a disposable line, leads through the cover 1′ into the chamber 1. A second container 2 forms an underwater seal and is connected to the container 1 via the connecting line 4. The underwater seal filled partially with water forms a kind of nonreturn valve in which air or fluid is led off but cannot flow back to the patient.

The container 2, likewise closed tight with the cover 2′, is connected via a line 6 to a vacuum pump 5 which for its part is operated by a battery 7 (for stationary use, the system can alternately be plugged into the mains). A control unit 8 is provided for operating the system.

To improve the mobility of a patient, all the system components are arranged on a mobile trolley so that, by virtue of the battery operating, complete independence is ensured. The trolley can of course also receive infusion containers.

The drainage line 3 (disposable article), and the container 1 (secretion-collecting chamber) which can be cleaned, are to be replaced from time to time.

The drainage system can not only be used for thorax drainage but for drainage of fluids from all kind of human body cavities, such as for gastric drainage. In this case however, the underwater seal is usually not necessary.

What is claimed is:

1. A thorax drainage system, characterized by the combination of the following components:
a first container forming a secretion-collecting chamber;
a separate second container forming a nonreturn valve in the form of an underwater seal;
a drainage line which can be attached to a patient and leads into the secretion-collecting chamber;
a connecting line between the two containers;
a vacuum pump;
a connecting line between the vacuum pump and the container forming the underwater seal;
a battery for mains-free operation of the vacuum pump, and
a mobile trolley for receiving all the system components,
in which system the two containers can be closed tight with associated covers into which the lines open, and at least the drainage line is a disposable line.

2. The thorax drainage system as claimed in claim 1, characterized in that the two separate containers are reusable containers.

3. A thorax drainage system with a vacuum pump, a battery for mains-free operation of the vacuum pump, an underwater seal connected to the vacuum pump via a first connecting line, a secretion-collecting chamber connected to the underwater seal via a second connecting line, and a drainage line which can be attached to a patient and leads into the secretion-collecting container, said vacuum pump, battery, underwater seal and secretion-collecting chamber forming a mobile system.

4. The system as claimed in claim 3, in which the vacuum pump, the battery, the underwater seal and the secretion-collecting chamber are arranged on a mobile trolley.

5. The system as claimed in claim 4, in which infusion containers can also be received on the trolley.

6. The system as claimed in claim 4, in which the trolley is designed to receive all the components of the system.

7. The system as claimed in claim 3, in which the secretion-collecting chamber is a first container, and the underwater seal is a second, separate container forming a nonreturn valve.

8. The system as claimed in claim 7, in which the two containers can be closed tight with associated covers into which the first and second connecting lines and the drainage line open.

9. The system as claimed in claim 3, in which at least the drainage line is a disposable line.

10. The system as claimed in claim 7, in which the two separate containers are reusable containers.

11. A drainage system with a vacuum pump, a battery for mains-free operation of the vacuum pump, a secretion-collecting chamber and a connecting line for connecting the secretion-collecting chamber to the vacuum pump, and a drainage line which can be attached to a patient and leads into the secretion-collecting container, in which system the vacuum pump, the battery and the secretion-collecting chamber are arranged on a mobile trolley.

12. The system as claimed in claim 11, in which infusion containers can also be received on the trolley.

13. The system as claimed in claim 11, in which the trolley is designed to receive all the components of the system.

14. The system as claimed in claim 11, in which the secretion-collecting chamber is a container and can be closed tight with an associated cover, and the connecting line and the drainage line open into the cover.

15. The system as claimed in claim 11, in which at least the drainage line is a disposable line.

16. The system as claimed in claim 14, in which the container is a reusable container.
